(19) United States
(12) Patent Application Publication
(10) Pub. No.: US 2016/0312258 A1
(43) Pub. Date: Oct. 27, 2016

(54) METHOD FOR PRODUCING FERMENTATION-RAW-MATERIAL SUGAR SOLUTION AND METHOD FOR PRODUCING CHEMICAL OBTAINED BY FERMENTING THE FERMENTATION-RAW-MATERIAL SUGAR SOLUTION

(71) Applicant: JGC CORPORATION, Tokyo (JP)

(72) Inventors: MAKOTO IKEO, IBARAKI (JP); SHOHEI OKINO, IBARAKI (JP); DAISUKE TANEDA, IBARAKI (JP)

(73) Assignee: JGC CORPORATION, TOKYO (JP)

(21) Appl. No.: 15/101,900

(22) PCT Filed: Dec. 6, 2013
(86) PCT No.: PCT/JP2013/082805

§ 371 Date: Jun. 4, 2016

(51) Int. Cl. C12P 19/14; C12P 19/02; C12P 7/14

(52) U.S. Cl. CPC C12P 19/14; C12P 7/14; C12P 19/02; C12P 2201/00; C12P 2203/00

(57) ABSTRACT

A method for producing a fermentation-raw-material sugar solution includes a step of saccharifying non-food biomass to obtain a low-concentration-sugar-containing solution, and a step of mixing the low-concentration-sugar-containing solution with a sugar or high-concentration-sugar-containing solution derived from food biomass to obtain a fermentation-raw-material sugar solution.

### TECHNICAL FIELD

The present invention relates to a method for producing a fermentation-raw-material sugar solution and a method for producing a chemical obtained by fermenting the fermentation-raw-material sugar solution.

### BACKGROUND ART

In recent years, for the purpose of using an energy source that replaces the petroleum resource, studies have been performed on a method of enzymatically producing a sugar solution from woody or herbaceous materials, for example, non-food biomass such as woodchips and rice straw and fermenting the sugar to produce an alcohol (for example, ethanol), as described in Patent Literature 1, for example.

There has been a demand for the widespread use of a method of enzymatically saccharifying appropriately pretreated non-food biomass to produce a sugar solution.

At present, commercialization of a process of fermenting this sugar solution with yeast or the like to produce ethanol is underway. However, in order to achieve widespread use, further enhancement of the cost effectiveness is required.

The sugar solution, which is used to produce ethanol, is also expected to be used to produce various chemicals such as butanol, succinic acid, and lactic acid.

When ethanol or other various chemicals are produced, a sugar solution serving as the raw material desirably has a concentration of about 20 to about 30% by weight. Below this concentration, fermentation product in the fermentation culture solution is produced at a low concentration and recovery of the fermentation product requires energy, which is problematic. Above that concentration, fermentation microorganisms are inhibited, resulting in inefficient fermentation.

However, when non-food biomass alone is used as the raw material to produce a high-concentration sugar solution by an existing method, the reaction tank has a high solid-liquid ratio (biomass concentration) and a large amount of enzyme is required. This results in an increase in the production cost of the sugar solution, which has been problematic.

In order to reduce the amount of enzyme used, for example, enzyme-producing microorganisms have been developed. Mass production of the enzyme has contributed to reduction in the cost and enhancement of enzyme activity has contributed to reduction in the amount of enzyme used.

### CITATION LIST

### Patent Literature

PTL 1: Japanese Unexamined Patent Application Publication No. 2006-87319

### SUMMARY OF INVENTION

### Technical Problem

However, the development of enzyme-producing microorganisms alone is insufficient for establishing the technique of inexpensively producing a sugar solution. That is, the establishment requires the development of a method for producing a sugar solution, the method enabling reduction in the amount of enzyme used.

From the viewpoint of the method for producing a sugar solution, in order to reduce the amount of enzyme used, a method of producing a sugar solution so as to have a low sugar concentration may be considered. This is because, as the sugar concentration increases, the enzyme is inhibited by the sugar, resulting in a decrease in the activity.

On the other hand, when the sugar solution is thus produced so as to have a low concentration and used as a raw material to produce ethanol or other various chemical materials by fermentation, large-scale fermentation facilities are used and the fermentation product is produced at a low concentration. Thus, facilities for concentrating and purifying this product and the operating cost result in increases in the cost.

Under the above-described circumstances, the present invention has been accomplished. An object of the present invention is to provide a method for producing a fermentation-raw-material sugar solution, the method enabling reduction in the total cost such as reduction in the amount of enzyme used and reduction in the facility cost; and to provide a method for producing a chemical obtained by fermenting the fermentation-raw-material sugar solution.

### Solution to Problem

A first aspect of the present invention is a method for producing a fermentation-raw-material sugar solution, the method including a step of saccharifying non-food biomass to obtain a low-concentration-sugar-containing solution, and a step of mixing the low-concentration-sugar-containing solution with a sugar or high-concentration-sugar-containing solution derived from food biomass to obtain a fermentation-raw-material sugar solution.

A second aspect of the present invention is a method for producing a chemical, the method including a step of producing a fermentation-raw-material sugar solution in accordance with the first aspect; and a step of fermenting the fermentation-raw-material sugar solution obtained by the step to produce a chemical.

### Advantageous Effects of Invention

A method for producing a fermentation-raw-material sugar solution according to the present invention can provide a method for producing a fermentation-raw-material sugar solution, the method enabling reduction in the total cost such as reduction in the amount of enzyme used and reduction in the facility cost, and can provide a method for producing a chemical obtained by fermenting the fermentation-raw-material sugar solution.

### BRIEF DESCRIPTION OF DRAWINGS

FIG. 1 illustrates a conventional technique with respect to the present invention.

FIG. 2 illustrates an example of an embodiment according to the present invention.

FIG. 3 is a graph indicating experimental results of enzymatic saccharification of steam-exploded bagasse and hemicellulose-removed bagasse containing cellulose as a main component.

### DESCRIPTION OF EMBODIMENTS

A method for producing a fermentation-raw-material sugar solution and a method for producing a chemical obtained by fermenting the fermentation-raw-material sugar solution according to embodiments of the present invention will be described.

Note that the embodiments are described to provide specific explanations for better understanding of the gist of the invention and do not limit the present invention unless otherwise specified.

### <<Method for Producing Fermentation-Raw-Material Sugar Solution>>

A first aspect of the present invention is a method for producing a fermentation-raw-material sugar solution, the method including a step of saccharifying non-food biomass to obtain a low-concentration-sugar-containing solution, and a step of mixing the low-concentration-sugar-containing solution with a sugar or high-concentration-sugar-containing solution derived from food biomass to obtain a fermentation-raw-material sugar solution.

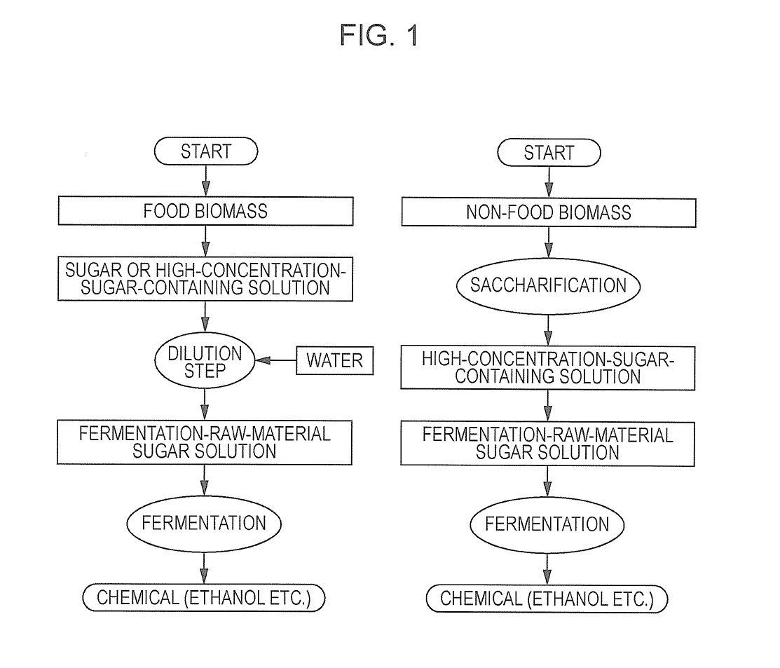

Conventionally, as illustrated in FIG. 1, a step of obtaining a sugar-containing solution derived from non-food biomass and a step of obtaining a sugar-containing solution or the like derived from food biomass have been independent from each other. Accordingly, facilities have also been independently built, such as fermentation tanks and distillation columns that use the fermentation-raw-material sugar solutions as raw materials to produce various chemicals such as alcohol.

The method for obtaining a sugar-containing solution derived from non-food biomass has been conventionally performed. In the conventional method, a sugar solution having a high concentration of 15% by weight or more has been required when the sugar-containing solution is fermented to produce various chemicals such as alcohol, in order to enhance the cost effectiveness of the conversion step such as a distillation step to achieve commercialization.

However, in order to prepare a sugar-containing solution so as to have a high sugar concentration, the amount of enzyme used increases. Thus, the use of a large amount of expensive enzyme makes it difficult to establish the technique of inexpensively producing a sugar solution.

On the other hand, in the step of obtaining a sugar-containing solution or the like derived from food biomass, the following two processes are representative processes, for example:

(1) a process of producing ethanol or other chemicals directly from sugar cane juice; and

(2) a process of producing sugar from sugar cane juice and using the by-product, blackstrap molasses, as raw material to produce ethanol or other chemicals.

In the process (1) above, sugar cane juice is concentrated during which impurities are removed and the juice is concentrated, to thereby facilitate temporary storage. In this case, sugar cane juice is concentrated such that the sugar concentration becomes about 40% by weight; and, in the actual use of the sugar solution as a fermentation raw material, the sugar solution is diluted with water so as to have an appropriate concentration.

In the process (2) above, blackstrap molasses generated during production of sugar has a sugar concentration of about 50 to about 60% by weight. In the use of the blackstrap molasses as a fermentation raw material, the blackstrap molasses is diluted with water so as to have an appropriate concentration.

In the fermentation step using a sugar-containing solution derived from food biomass, the inventors of the present invention have directed attention to the above-described dilution step. Specifically, the inventors have conceived replacement of the conventionally used dilution water by a non-food-derived low-concentration-sugar-containing solution. By using a non-food-derived low-concentration-sugar-containing solution as a substitute for the dilution water, even when the non-food-derived sugar solution has a low concentration, a fermentation-raw-material sugar solution having a target sugar concentration can be produced. As described above, production of a non-food-derived high-concentration sugar solution involves an increase in the amount of enzyme used. In contrast, use of a non-food-derived low-concentration-sugar-containing solution as a substitute for dilution water enables reduction in the amount of enzyme used during the production of the non-food-derived sugar solution. Thus, reduction in the cost is achieved and the non-food-derived low-concentration-sugar-containing solution can be effectively used.

Employment of this method eliminates the necessity of producing a high-concentration saccharified solution from non-food biomass as the raw material with an increased amount of enzyme. In other words, while a small amount of enzyme is used to saccharify non-food biomass, a high-concentration sugar solution suitable as fermentation raw material can be produced.

In addition, the step of obtaining a sugar-containing solution derived from non-food biomass is integrated with the step of obtaining a sugar-containing solution or the like derived from food biomass, the steps having required independent facilities conventionally. As a result, the steps can share facilities such as fermentation facilities, product recovery and purification facilities, and waste fluid treatment facilities, which enables reduction in the facility cost and energy cost.

The present invention is based on the unprecedented breakthrough idea and relates to the original technical idea.

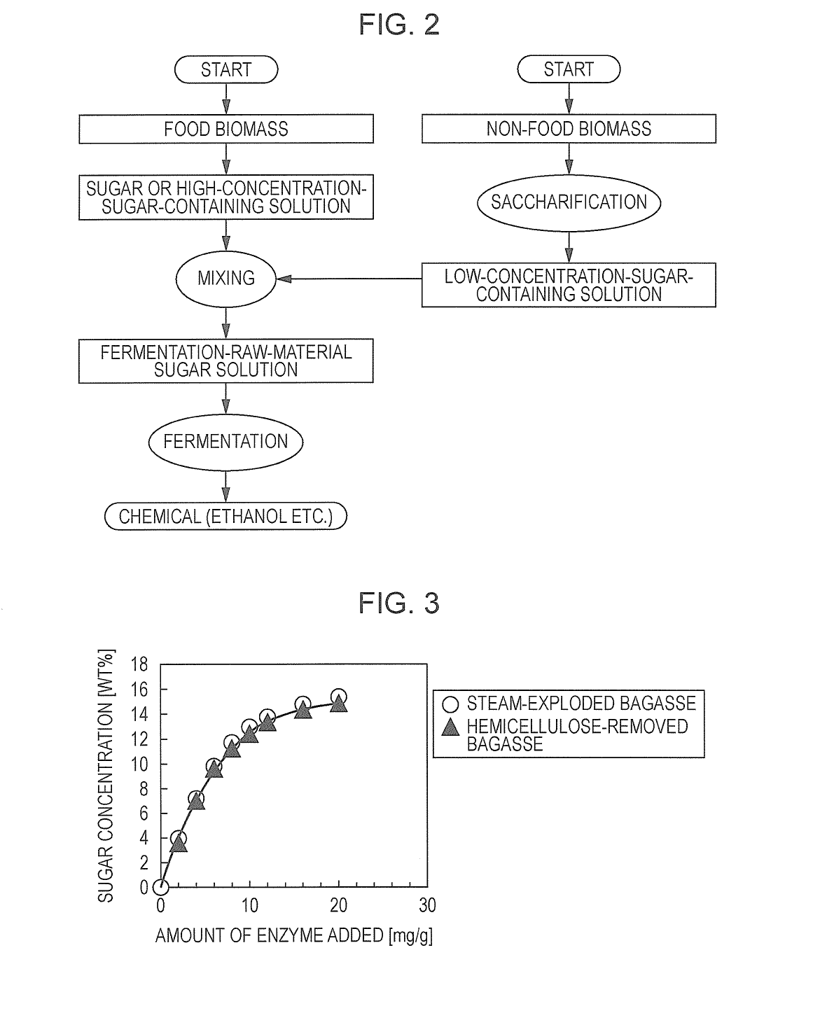

Hereinafter, a first aspect according to the present invention will be described with reference to FIG. 2.

### [Step of Saccharifying Non-Food Biomass to Obtain Low-Concentration-Sugar-Containing Solution]

As illustrated in FIG. 2, this embodiment includes a step of saccharifying non-food biomass to obtain a low-concentration-sugar-containing solution.

In the present invention, specific examples of the non-food biomass include herbaceous biomass such as bagasse, corn stover, corncobs, switchgrass, napier grass, Erianthus, rice straw, and wheat straw, and woody biomass such as trees and building waste. Cellulose and hemicellulose, which are polysaccharide components, in non-food biomass are hydrolyzed to provide a sugar solution that contains monosaccharides and is usable as a fermentation raw material.

In the embodiment, the non-food biomass may be prepared by removing hemicellulose from bagasse, corn stover, or corncobs so as to contain cellulose as a main component.

When bagasse, corn stover, or corncobs are degraded with dilute sulfuric acid, organic acid, hot water, or steam, hemicellulose in the biomass is degraded into xylose or furfural and separated from the biomass.

Xylose is a pentose and used for processing food or used as a raw material for producing xylitol. Furfural, which is one of aromatic aldehydes, is a compound used as a raw material for various chemical products such as solvents and synthetic rubbers. During production of xylose or furfural, hemicellulose in non-food biomass is converted into xylose or furfural and residue after recovery of xylose or furfural is discharged. In the embodiment, as the non-food biomass, the “biomass prepared by removing hemicellulose so as to contain cellulose as a main component” is preferably the residue after recovery of xylose or furfural.

In addition, during enzymatic saccharification of biomass, removal of hemicellulose in advance facilitates enzymatic saccharification. Accordingly, the method of removing hemicellulose with dilute sulfuric acid, organic acid, hot water, or steam is also employed as a pretreatment method of enzymatic saccharification of biomass. In the embodiment, as the “biomass prepared by removing hemicellulose so as to contain cellulose as a main component”, the biomass prepared by removing hemicellulose is also preferably employed for the above-described reason.

In the embodiment, prior to reaction between non-food biomass and the enzyme, a predetermined pretreatment may be performed. The pretreatment means that non-food biomass is subjected to physical or chemical treatment. Non-limiting specific examples of the pretreatment include a steam-explosion treatment (cooking-explosion treatment of performing cooking with steam and instantaneously releasing the pressure to cause rupture due to volume expansion); an acid treatment using high-temperature high-pressure dilute sulfuric acid, sulfite, or the like; an alkali treatment using an alkaline aqueous solution such as calcium hydroxide or sodium hydroxide; an ammonia treatment using liquid ammonia, ammonia gas, or aqueous ammonia; a hydrothermal treatment using pressurized hot water; and a pulverizing treatment of mechanically cutting fibers with a cutter mill, a hammer mill, a grinder, or the like.

In the embodiment, the sugar-containing solution is an aqueous solution containing monosaccharide of glucose and/or xylose and oligosaccharide in water, the aqueous solution being prepared by saccharifying non-food biomass directly or pretreated non-food biomass to hydrolyze cellulose, or cellulose and hemicellulose in non-food biomass.

In the embodiment, the saccharification treatment is not particularly limited and may be, for example, an enzymatic saccharification treatment using an enzyme.

In the embodiment, the saccharifying enzyme (cellulase) used for the enzymatic saccharification treatment is an enzymatic component that has the activity of degrading cellulose or cellulose and hemicellulose, or that assists degradation of cellulose or cellulose and hemicellulose. Specific examples of the enzymatic component include cellobiohydrolase, endoglucanase, β-glucosidase, xylanase, xylosidase, and a biomass swelling enzyme. The saccharifying enzyme is preferably an enzyme mixture containing plural components among the components. The concerted effect or the complementary effect provided by such plural enzyme components enables, for example, efficient hydrolysis of cellulose and hemicellulose. Accordingly, plural enzyme components are preferably used in the embodiment.

In the embodiment, the sugar-containing solution derived from non-food biomass has a low concentration.

As described above, in the embodiment, the sugar-containing solution derived from non-food biomass is used as a substitute for water conventionally used as a concentration adjusting liquid or a dilution liquid, in the [step of mixing with a sugar or high-concentration-sugar-containing solution derived from food biomass to obtain a fermentation-raw-material sugar solution] described below. Accordingly, the sugar-containing solution is not particularly limited as long as it has a low concentration; the concentration is preferably 5 to 12% by weight.

In the embodiment, by preparing the sugar-containing solution derived from non-food biomass so as to have a low concentration, the amount of enzyme used can be reduced, which contributes to reduction in the production cost.

In the embodiment, in the [step of saccharifying non-food biomass to obtain a low-concentration-sugar-containing solution], biomass of the embodiment or biomass pretreated by a pretreatment method is produced.

Next, the biomass and an aqueous solution (enzyme aqueous solution) containing an appropriate amount of cellulase suitable for degradation of cellulose and/or hemicellulose in the biomass are placed into a reaction tank (enzymatic degradation tank), and the biomass and the enzyme aqueous solution are mixed (preparation step).

In this preparation step, the pH of the reaction tank solution is adjusted such that the pH of the reaction tank solution satisfies the pH condition optimal for the enzyme used. In addition, the temperature of the reaction tank is adjusted so as to satisfy the temperature condition optimal for the enzyme used.

In this preparation step, the pH of the mixture of biomass, enzyme aqueous solution, and additives is preferably adjusted such that the enzyme actively functions; specifically, the pH of the reaction-system aqueous solution is preferably adjusted to be 4 to 6.

In this preparation step, the temperature of the mixture is preferably adjusted such that the enzyme actively functions; specifically, the temperature of the reaction system is preferably increased to 40 to 60° C.

The concentration of biomass within the reaction tank is preferably 5 g to 50 g relative to 100 mL of the solution, that is, 5 w/v % to 50 w/v %, more preferably 10 g to 30 g relative to 100 mL of the solution, that is, 10 w/v % to 30 w/v %.

An enzyme used to degrade biomass is cellulase.

When biomass has a high hemicellulose content, in addition to cellulase, an enzyme that degrades hemicellulose such as xylanase or mannanase is preferably added.

In order to stir the mixture, a stirring impeller is used, for example.

In the embodiment, the mixture is gently mixed by stirring within the reaction tank such that the enzyme contained in the enzyme aqueous solution is not excessively inactivated, to thereby cause efficient enzymatic saccharification of biomass (cellulose and/or hemicellulose).

In this enzymatic saccharification reaction step, the temperature of the mixture is preferably adjusted such that the enzyme actively functions; specifically, the temperature is preferably maintained at 40 to 60° C.

The enzymatic saccharification reaction step is performed until enzymatic saccharification of biomass has sufficiently proceeded and the reaction no longer proceeds; for example, enzymatic degradation of biomass is performed at 40 to 60° C. for about 2 to about 20 days.

[Step of Mixing with Sugar or High-Concentration-Sugar-Containing Solution Derived from Food Biomass to Obtain Fermentation-Raw-Material Sugar Solution]

In the embodiment, the high-concentration-sugar-containing solution derived from food biomass is preferably blackstrap molasses, sugar cane juice, or concentrated sugar cane juice.

In the embodiment, the blackstrap molasses is a by-product generated during sugar production from juice or raw sugar of sugar cane or sugar beet. In other words, in the crystallization step during sugar production, the blackstrap molasses is the residual solution containing sugar components after crystallization.

In the embodiment, the sugar derived from food biomass is preferably sugar derived from starch obtained from corn or cassava as the raw material.

In the embodiment, as illustrated in FIG. 2, for example, when sugar cane is used as the food biomass, in order to enhance storability of sugar cane juice or remove impurities, the sugar cane juice is concentrated to provide a 40 to 60% by weight high-concentration-sugar-containing solution.

Alternatively, for example, when blackstrap molasses is used as the food biomass, blackstrap molasses generally has a concentration of 50 to 60% by weight, which is a high-concentration-sugar-containing solution.

In the embodiment, as illustrated in FIG. 2, in order to adjust the high-concentration-sugar-containing solution so as to have a concentration suitable for fermentation, the low-concentration-sugar-containing solution obtained in the [step of saccharifying non-food biomass to obtain a low-concentration-sugar-containing solution] is mixed to prepare a fermentation-raw-material sugar solution. The mixing method is not particularly limited and may be a known method.

### <<Method for Producing Chemical>>

A method for producing a chemical obtained by fermenting a fermentation-raw-material sugar solution according to a second aspect of the present invention is a method for producing a chemical, the method including a step of producing a fermentation-raw-material sugar solution by the above-described method for producing a fermentation-raw-material sugar solution, and a step of fermenting the fermentation-raw-material sugar solution obtained by the step to produce a chemical.

The [step of producing a fermentation-raw-material sugar solution by the method for producing a fermentation-raw-material sugar solution] is the same as above.

### [Step of Fermenting Fermentation-Raw-Material Sugar Solution to Produce Chemical]

A microorganism having the capability of producing a chemical from a sugar-containing solution obtained by the method for producing a fermentation-raw-material sugar solution according to the first aspect of the present invention is incubated by fermentation, to thereby produce the chemical. The fermentation-raw-material sugar solution obtained in the present invention contains, as the main component, glucose and/or sucrose serving as a carbon source for growing microorganisms or mold, so that it can be effectively used as the fermentation raw material, in particular, a carbon source.

Examples of the microorganism or mold used in the method for producing a chemical obtained by fermenting a fermentation-raw-material sugar solution in the present invention include yeast such as bakers' yeast, which is commonly used in the fermentation industry, bacteria such as colon bacilli and corynebacteria, filamentous fungi, and actinomycetes. Such microorganisms or mold used may be those isolated from the natural environment or those modified in terms of some properties by mutation or genetic recombination.

In the present invention, a fermentation liquid used in the method for producing a chemical is preferably a fermentation liquid that contains a sugar solution and further appropriately contains a nitrogen source, mineral salts, and optionally organic micronutrients such as amino acids and vitamins. The sugar solution of the present invention contains, as a carbon source, a monosaccharide usable by the microorganism or mold, such as glucose or sucrose. In some cases, as other carbon sources, for example, saccharides such as xylose, fructose, galactose, and lactose, saccharified starch solutions containing such saccharides, sweet potato molasses, sugar beet molasses, hi-test molasses, organic acids such as acetic acid, alcohols such as ethanol, or glycerin may be added and the resultant solution may be used as the fermentation raw material. Examples of the nitrogen source include ammonia gas, aqueous ammonia, ammonium salts, urea, nitrates, other auxiliary organic nitrogen sources such as oil cakes, soybean hydrolysate solution, casein hydrolysate, other amino acids, vitamins, corn steep liquor, yeast or yeast extract, meat extract, peptides such as peptone, various fermenting microorganisms, and hydrolysate thereof. As the mineral salts, for example, phosphate, magnesium salt, calcium salt, iron salt, or manganese salt can be appropriately added.

When a microorganism or mold used in the present invention requires specific nutrients for growth, the nutrients may be added in the form of a preparation or a natural product containing the nutrients. An antifoaming agent may be optionally used.

Normally, the fermentation with a microorganism or mold is performed under conditions of a pH of 4 to 8 and a temperature of 20 to 40° C. Normally, the pH of the culture solution is adjusted to a predetermined value within the range of 4 to 8 with inorganic or organic acid, alkaline material, urea, calcium carbonate, or ammonia gas, for example. When the rate of supplying oxygen needs to be increased, for example, oxygen may be added to air so as to maintain an oxygen concentration of 21% or more, the culture may be pressurized, the stirring rate may be increased, or the flow rate of air may be increased.

As the method for producing a chemical in which the sugar solution obtained by the method for producing a sugar solution according to the present invention is used as the fermentation raw material, a fermentation incubation method known to those skilled in the art is employed.

Examples of the chemical produced by fermenting the fermentation-raw-material sugar solution in the embodiment include mass-produced materials in the fermentation industry such as alcohols, organic acids, amino acids, and nucleic acids. Specific examples include ethanol, methanol, propanol, isopropanol, butanol, isobutanol, ethylene glycol, propanediol, butanediol, glycerin, erythritol, xylitol, sorbitol, acetic acid, lactic acid, propionic acid, 3-hydroxypropionic acid, butyric acid, gluconic acid, itaconic acid, citric acid, succinic acid, levulinic acid, glutamic acid, aspartic acid, methionine, lysine, glycine, arginine, threonine, phenylalanine, tyrosine, methane, ethylene, acetone, and industrial enzymes.

### EXAMPLES

Hereinafter, the present invention will be more specifically described with reference to Experimental Example and Examples. However, the present invention is not limited to the following Experimental Example.

### Experimental Example

The non-food biomass used was bagasse and bagasse prepared by removing hemicellulose so as to contain cellulose as the main component.

The bagasse was pretreated by steam-explosion. The steam-explosion was performed by cooking at 230° C. for 5 minutes.

The bagasse prepared by removing hemicellulose so as to contain cellulose as the main component, was prepared by treating bagasse with 0.5% dilute sulfuric acid at 170° C. for 3 minutes to remove hemicellulose.

To the pretreated bagasse and the bagasse prepared by removing hemicellulose so as to contain cellulose as the main component, a saccharifying enzyme (cellulase) was added in amounts of enzyme added, which are described in Table 1, and saccharification was performed under the following conditions.

Vessel: baffled shake flask 250 mL

Temperature: 50° C.

pH: 5

Shaking rate: 120 rpm

Table 1 also describes the relationship between the amount of saccharifying enzyme added and the corresponding biomass concentration and sugar concentrations in Experimental Example. The amount of enzyme is described as the amount of protein per dry weight of the biomass.

TABLE 1

| | | Sugar | Sugar |
|---|---|---|---|
| Amount of | | Concentration | Concentration |
| Enzyme | Biomass | (Steam-Exploded | (Hemicellulose- |
| Added | Concentration | Bagasse) | Removed Bagasse) |
| mg/g | w/v % | wt % | wt % |
| 0 | 0 | 0 | 0 |
| 2 | 10 | 4.0 | 3.6 |
| 4 | 15 | 7.3 | 7.1 |
| 6 | 20 | 9.9 | 9.7 |
| 8 | 25 | 11.8 | 11.3 |
| 10 | 30 | 13.0 | 12.5 |
| 12 | 33 | 13.8 | 13.4 |
| 16 | 37 | 14.8 | 14.4 |
| 20 | 40 | 15.4 | 14.9 |

The enzymatic saccharification experiment was performed under the conditions of Experimental example, and the concentrations of sugar solutions at the fifth day from the initiation of the reaction were measured.

The results are described in FIG. 3. The results indicate that the relationship between the sugar concentration and the amount of enzyme added was substantially the same in both of the case where the raw material was the pretreated bagasse and the case where the raw material was the bagasse prepared by removing hemicellulose so as to contain cellulose as the main component. In order to produce a high-concentration sugar solution, as the amount of enzyme added is increased and the biomass concentration is increased, the amount of enzyme added and the sugar concentration increase in a linear relationship up to a sugar concentration of about 12% by weight; however, at this concentration or higher, a very large amount of enzyme is required to increase the sugar concentration. In other words, when a sugar solution having a sugar concentration of about 12% by weight or more is produced, the effect of sugar inhibition is strongly exhibited and the enzyme activity is inhibited. Stated another way, in order to efficiently (inexpensively) produce a sugar solution, the sugar concentration needs to be suppressed to about 12% by weight or less.

### Example 1

### Amount of Enzyme Used in Step of Mixing Non-Food Biomass and Food Biomass

In the step of mixing non-food biomass and food biomass, the sugar-containing solution derived from non-food biomass is used for mixing with a sugar or high-concentration-sugar-containing solution derived from food biomass, and hence is prepared so as to have a sugar concentration of 12% by weight or less.

In order to achieve the sugar concentration of 12% by weight, the following conditions were required.

Amount of enzyme added: 8 mg/g-substrate

Biomass concentration: 25 w/v %

On the basis of this experiment result, the amount of enzyme required for producing 1 g of sugar was determined as below by calculation.

When the sugar concentration is 12% by weight: 17 mg

A 40% by weight high-concentration-sugar-containing solution derived from food biomass was mixed with a sugar-containing solution that was derived from non-food biomass and had a sugar concentration of 12% by weight, to prepare a 20% by weight fermentation-raw-material sugar solution.

### Reference Example 1

### Amount of Enzyme Used in Step of Independently Obtaining Sugar-Containing Solution Derived from Non-Food Biomass

In the step of independently obtaining a sugar-containing solution from non-food biomass, in consideration of the conversion step such as a distillation step, a sugar-containing solution having a sugar concentration of 15% by weight was prepared.

In order to achieve the sugar concentration of 15% by weight, the following conditions were required.

Amount of enzyme added: 16 mg/g-substrate

Biomass concentration: 37 w/v %

On the basis of this experiment result, the amount of enzyme required for producing 1 g of sugar was determined as below by calculation.

When the sugar concentration is 15% by weight: 30 mg

Thus, compared with the case of producing the high-concentration sugar solution (15% by weight: Reference Example 1), the amount of enzyme used was only 17/30=about 57% in the case of producing the low-concentration sugar solution (12% by weight: Example 1). In other words, it has been found that Example 1 enables 43% reduction in the amount of enzyme used.

### Example 2

### Production of Ethanol

The 20% by weight fermentation-raw-material sugar solution in Example 1 enables growth of microorganisms and enables efficient ethanol production by microorganisms.

### Comparative Example 1

### Method of Producing Ethanol

The 15% by weight fermentation-raw-material sugar solution in Reference Example 1 enables growth of microorganisms and enables production of ethanol by microorganisms. However, as described in Reference Example 1, compared with Example 2 using the fermentation-raw-material sugar solution of Example 1, an additional amount of enzyme as much as 43% is required, so that efficient production cannot be achieved.

### INDUSTRIAL APPLICABILITY

The present invention relates to use of a mixture of food-based sugar and non-food-based sugar. From the viewpoint of the current enzyme performance and enzyme price, it is difficult to commercialize production of a high-concentration non-food sugar solution. However, when mixing with food-based sugar is employed, production of a low-concentration non-food-based sugar solution will suffice, so that use of non-food-based sugar is promoted. When the use of non-food-based sugar is thus promoted, the global environmental issues and the food problem can be addressed.

1. A method for producing a fermentation-raw-material sugar solution, comprising:
a step of saccharifying non-food biomass to obtain a low-concentration-sugar-containing solution, and
a step of mixing the low-concentration-sugar-containing solution with a sugar or high-concentration-sugar-containing solution derived from food biomass to obtain a fermentation-raw-material sugar solution.

2. The method for producing a fermentation-raw-material sugar solution according to claim 1, wherein the low-concentration-sugar-containing solution has a sugar concentration of 5 to 12% by weight.

3. The method for producing a fermentation-raw-material sugar solution according to claim 1, wherein the sugar or high-concentration-sugar-containing solution derived from food biomass is blackstrap molasses or sugar cane juice.

4. The method for producing a fermentation-raw-material sugar solution according to claim 1, wherein the sugar or high-concentration-sugar-containing solution derived from food biomass is derived from starch obtained from corn or cassava as raw material.

5. The method for producing a fermentation-raw-material sugar solution according to claim 1, wherein the non-food biomass is bagasse, corn stover, or corncobs.

6. The method for producing a fermentation-raw-material sugar solution according to claim 1, wherein the non-food biomass is prepared by removing hemicellulose from bagasse, corn stover, or corncobs so as to contain cellulose as a main component.

7. A method for producing a chemical, comprising:
a first step of producing a fermentation-raw-material sugar solution by the method for producing a fermentation-raw-material sugar solution according to claim 1; and a second step of fermenting the fermentation-raw-material sugar solution obtained by the first step to produce a chemical.

8. The method for producing a chemical according to claim 7, wherein the chemical is ethanol.

9. The method for producing a chemical according to claim 7, wherein the chemical is succinic acid, butanol, butanediol, lactic acid, or cellulase.
